(19) United States
(12) Patent Application Publication
(10) Pub. No.: US 2007/0167774 A1
(43) Pub. Date: Jul. 19, 2007

(54) ULTRASOUND DIAGNOSTIC SYSTEM AND METHOD OF DETECTING LESION

(75) Inventors: Mok Kun Jeong, Seoul (KR); Sung Jae Kwon, Seoul (KR); Ra Young Yoon, Seoul (KR)

Correspondence Address: OBLON, SPIVAK, MCCLELLAND, MAIER & NEUSTADT, P.C., 1940 DUKE STREET, ALEXANDRIA, VA, 22314, US

(73) Assignee: Medison Co., Ltd., Hongchun-gun (KR)

(21) Appl. No.: 11/617,245

(22) Filed: Dec. 28, 2006

(30) Foreign Application Priority Data
Dec. 28, 2005 (KR) 10-2005-0131293

(51) Int. Cl. A61B 8/00

(52) U.S. Cl. 600/439

(57) ABSTRACT

An ultrasound diagnostic system and method of detecting thermal diffusion due to injection of high intensity focused ultrasound (HIFU) are disclosed. According to the embodiments of the present invention, first receive signals are obtained by ultrasound echo signals from the lesion that did not undergo the HIFU and second receive signals are obtained by ultrasound echo signal from the lesion that underwent the HIFU. Center frequencies of the first and second receive signals are detected, while center frequency shift between the first and second receive signals are calculated. A center frequency shift image is formed with the center frequency shift.

The present application claims priority from Korean Patent Application No. 10-2005-131293 filed on Dec. 28, 2005, the entire subject matter of which is incorporated herein by reference.

### BACKGROUND

1. Field

The present invention generally relates to an ultrasound diagnostic system and method, and more particularly to an ultrasound diagnostic system and method for detecting a lesion using high intensity focused ultrasound.

2. Background

An ultrasound diagnostic system has become an important and popular diagnostic tool due to its wide range of applications. Specifically, due to its non-invasive and non-destructive nature, the ultrasound diagnostic system has been extensively used in the medical profession. Modern high-performance ultrasound diagnostic systems and techniques are commonly used to produce two or three-dimensional (2D or 3D) diagnostic images of a target object. The ultrasound diagnostic system generally uses a probe including an array transducer having a plurality of transducer elements to transmit and receive ultrasound signals. The ultrasound diagnostic system forms ultrasound images of the internal structures of the target object by electrically exciting the transducer elements to generate ultrasound pulses that travel into the target object. The ultrasound pulses produce ultrasound echoes since they are reflected from a discontinuous surface of acoustic impedance of the internal structure, which appears as discontinuities to the propagating ultrasound pulses. Various ultrasound echoes return to the array transducer and are converted into electrical signals, which are amplified and processed to produce ultrasound data for forming an image of the internal structure of the target object.

In particular, an ultrasound diagnostic system using a high intensity focused ultrasound (HIFU) to remove a lesion (e.g., malignant tumor) in a body has been known to produce excellent treatment effects in the medical field. The lesion is necrotized with heat generated by focusing the high intensity ultrasound onto the lesion so that the lesion can be removed in the body. Since the generated heat may destroy other normal tissues around of the lesion in the body, the treatment should be performed carefully while focusing the ultrasound on the lesion so as not to damage the normal tissues. Conventionally, the ultrasound diagnostic system provides an ultrasound image showing the lesion being removed by the HIFU. That is, an operator can observe the process of removing the lesion.

The HIFU significantly increases temperature of a region on which the HIFU is focused, and the necrosis begins at a focal point of the HIFU. Therefore, it is necessary to avoid focusing the HIFU on the normal tissues and to observe thermal diffusion in real time when the HIFU is injected on the lesion, so as not to damage the normal tissues. However, it is difficult to observe the thermal diffusion in the conventional ultrasound diagnostic system.

### BRIEF DESCRIPTION OF THE DRAWINGS

Arrangements and embodiments may be described in detail with reference to the following drawings in which like reference numerals refer to like elements and wherein:

FIG. 1 illustrates an ultrasound diagnostic system according to one embodiment of the present invention;

FIG. 2 illustrates a flowchart showing a process of detecting the lesion according to one embodiment of the present invention;

FIG. 3 is a graph showing two center frequencies;

FIG. 4 depicts an example of a center frequency shift image; and

FIG. 5 illustrates a flowchart showing a process of detecting the lesion according to another embodiment of the present invention.

### DETAILED DESCRIPTION

A detailed description may be provided with reference to accompanying drawings. One of ordinary skill in the art may realize that the following description is illustrative only and is not in any way limiting. Other embodiments of the present invention may readily suggest themselves to such skilled persons having the benefit of this disclosure.

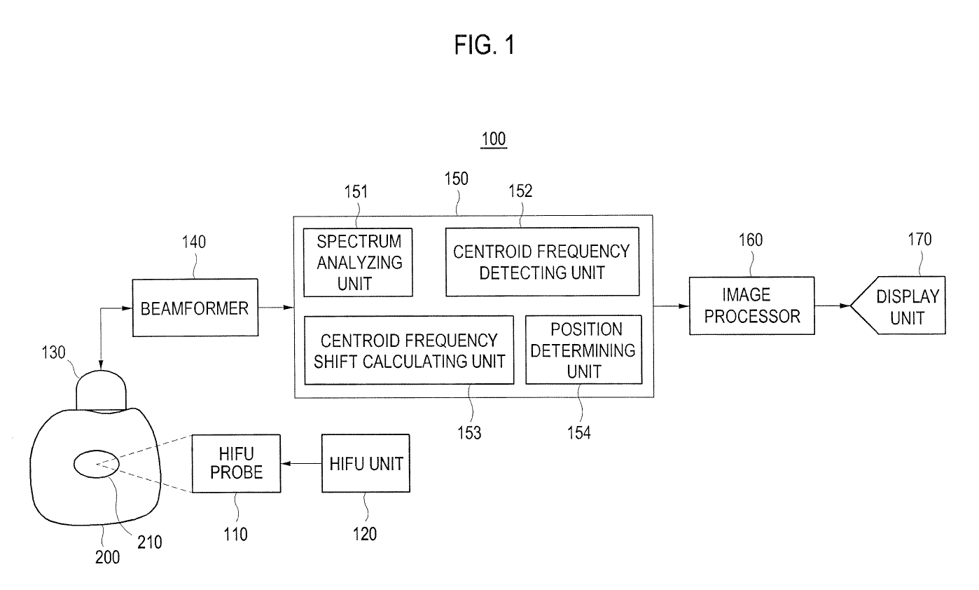

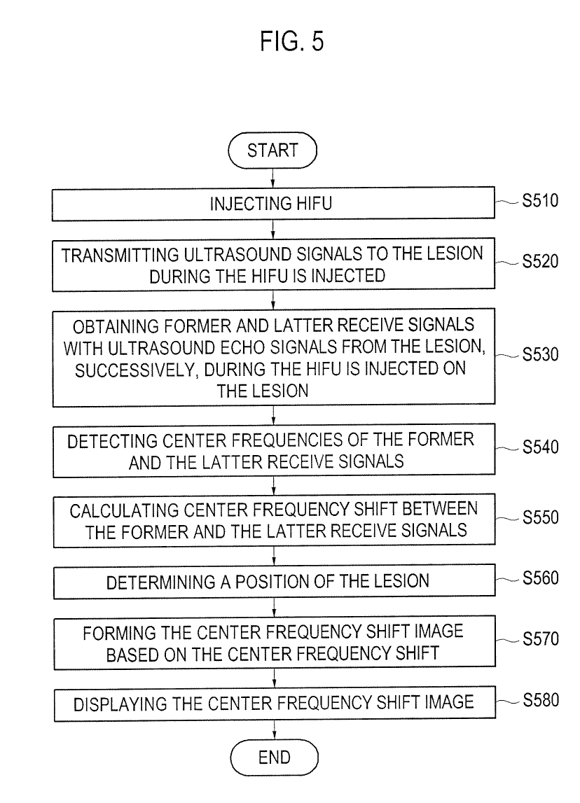

Hereinafter, embodiments of the present invention will be described with reference to FIGS. 1 and 5. The following embodiments describe detecting a lesion based on first receive signals and second receive signals. The first receive signals are obtained with ultrasound echo signals from the lesion, which has not undergone a high intensity focused ultrasound (HIFU). Further, the second receive signals are obtained with ultrasound echo signals from the lesion, which underwent the HIFU.

FIG. 1 illustrates an ultrasound diagnostic system according to one embodiment of the present invention. As shown in FIG. 1, an ultrasound diagnostic system 100 may include an HIFU probe 110, an HIFU unit 120, an imaging probe 130, a beamformer 140, a signal processing unit 150, an image processor 160 and a display unit 170. Further, the ultrasound diagnostic system 100 may include a container (not shown in FIG. 1) for containing a medium. The container may be a water tank, a water bag and the like. The container is disposed between the HIFU probe 110 and a body (i.e., skin) when the HIFU is injected into a body.

The HIFU probe 110 may inject the HIFU on a lesion 210 in a body 200. The HIFU probe 110 may include a number of elements. The HIFU unit 120 may provide the HIFU probe 110 with a high frequency power for generating the HIFU and control the HIFU probe 110 to inject the HIFU toward the lesion 210. As such, the HIFU unit 120 may include a power supply, a controller and a driver (not shown in FIG. 1). The power supply provides the high frequency power to the HIFU probe 110. The controller generates driving signals, and the driver drives the HIFU probe 110 according to the control signals outputted from the controller to focus the HIFU onto the lesion 210.

The imaging probe 130 may transmit ultrasound signals to the lesion 210 in the body 200 along transmit scanlines and receive ultrasound echo signals from the lesion 210. The imaging probe 130 may further output receive signals obtained from the ultrasound echo signals for forming an ultrasound image showing the lesion 210. The imaging probe 130 may include a number of elements arranged in a form of 1-dimensional or a 2-dimensional array. The imaging probe 130 may transmit ultrasound signals to a target object, namely, the lesion 210 in the body 200. The imaging probe 130 may receive ultrasound echo signals reflected from the lesion 210 and convert the ultrasound echo signals into the first and second receive signals.

The beamformer 140 may provide appropriate delays to the ultrasound signals for transmission such that the ultrasound signals outputted from the imaging probe 130 are focused on a focal point. Further, the beamformer 140 focuses the receive signals by considering the arrival time of the echo signals at each element in the imaging probe 130.

According to an embodiment of the present invention, the signal processing unit 150 may detect the center frequencies of two receive signals outputted from the imaging probe 130 and calculate the center frequency shift between the two receive signals. The signal processing unit 150 may include a spectrum analyzing unit 151, a center frequency detecting unit 152 and a center frequency shift calculating unit 153. The spectrum analyzing unit 151 may analyze a first spectrum and a second spectrum to produce spectrum analysis results. The first spectrum and the second spectrum are obtained from the first and the second receive signals. The center frequency detecting unit 152 may detect the first and second center frequencies from the analysis results, respectively. The center frequency shift calculating unit 153 may calculate the difference between the first and second center frequencies. In other words, the center frequency shift calculating unit 153 may calculate the shift of the first center frequency of the first receive signals due to the HIFU. The center frequency difference, namely, the center frequency shift, is a pixel value of each pixel for forming a center frequency shift image.

According to another embodiment of the present invention, the signal processing unit 150 may also determine a position of the lesion in consideration of the center frequency shift. In such a case, the signal processing unit 150 may further include a position determining unit 154. The position determining unit 154 may determine a position of the lesion based on the center frequency shift. Specifically, the position determining unit 154 may search a region in which pixels having pixel values within a predetermined range are adjacent to one another based on the fact that the center frequency shift is significant at the boundary of the lesion. The boundary of the region may be adopted to determine the position of the lesion boundary.

The image processor 160 may form at least one ultrasound image showing the lesion based on the receive signals and a center frequency shift image based on the center frequency shift. Also, the image processor 160 may form an overlap image of the ultrasound image and the center frequency shift image. Thus, the image processor 160 may remove the background of the center frequency shift image to form a transparent center frequency shift image, which shows only the boundary of the lesion, to be overlapped with the ultrasound image. Specifically, in order to form the transparent center frequency shift image, the image processor 160 may set the pixel values of all the pixels outside the region to zero in the center frequency shift image. According to another embodiment, the image processor 160 may express the background of the center frequency shift image with transparency in pseudo color map so as to leave only the boundary of the lesion.

The display unit 170 may display at least one of the ultrasound image, the center frequency shift image and the overlap image. For example, by dividing a display area of the display unit 170 into a plurality of sub-areas, the ultrasound image, the center frequency shift image and the overlap image may be displayed in each respective sub-areas.

Hereinafter, a method of detecting the lesion according to one embodiment of the present invention will be described with reference to FIGS. 1 to 4.

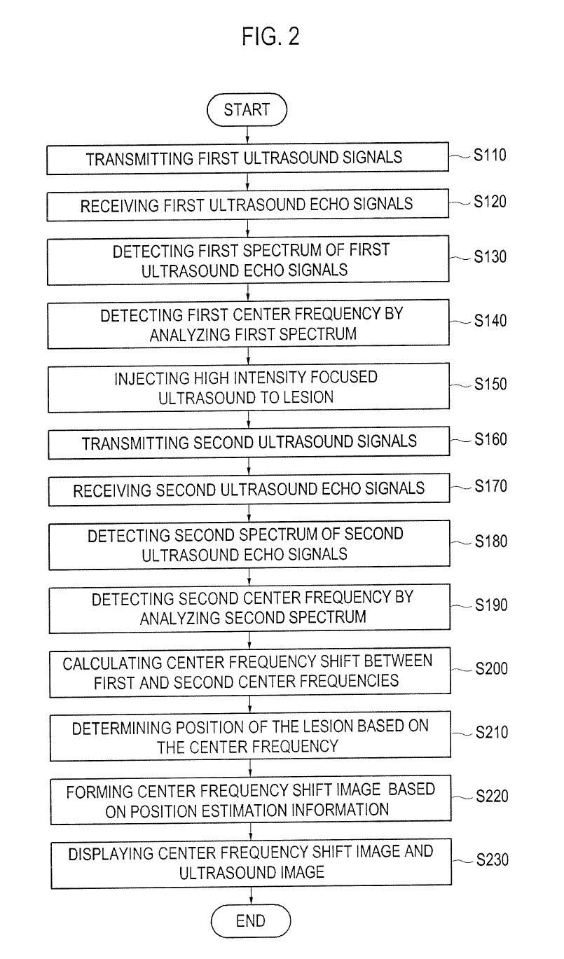

Referring now to FIGS. 1 and 2, the imaging probe 130 may transmit first ultrasound signals to a lesion in a body (step S110) and receive first ultrasound echo signals (step S120). The spectrum analyzing unit 151 may analyze the first spectrum of the first ultrasound echo signals to produce a first spectrum analysis result (step S130). Further, the center frequency detecting unit 152 may detect the first center frequency from the first spectrum based on the first spectrum analysis result (step S140).

The HIFU probe may inject the HIFU onto the lesion (step S150). Also, the imaging probe 130 may transmit second ultrasound signals to the lesion, which underwent the HIFU (step S160), and receive second ultrasound echo signals (step S170). The spectrum analyzing unit 151 may analyze a second spectrum of the second ultrasound echo signals to produce a second spectrum analysis result (step S180). The center frequency detecting unit 152 may detect a second center frequency from the second spectrum based on the second spectrum analysis result (step S190).

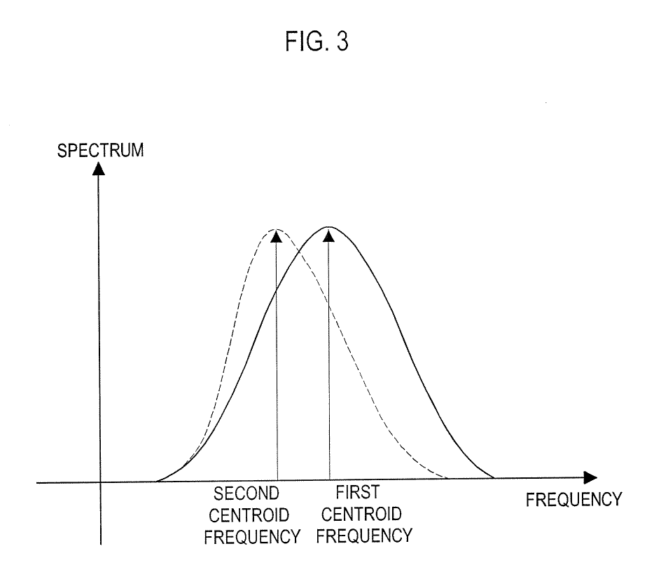

The center frequency shift calculating unit 153 may calculate a center frequency shift, that is, a difference between the first and second center frequencies as shown in FIG. 3 (step S200). The position determining unit 154 may determine the position of the lesion (i.e., boundary of the lesion) based on the center frequency shift calculated by the center frequency detecting unit 152 (step S210). The center frequency shift is a pixel value of each pixel for forming a center frequency shift image. After calculating the center frequency shift, the position determining unit 514 may search a region including pixels of which pixel values are within a predetermined range and adjacent to one another. In such a case, the position determining unit may determine the position of the lesion in consideration of a boundary of the region.

The image processor 160 may form the center frequency shift image based on the center frequency shift (step S220). The display unit 170 may display the center frequency shift image formed in the image processor 160 (step S220). In this embodiment, the steps 130 and 140 may be performed after the step 150.

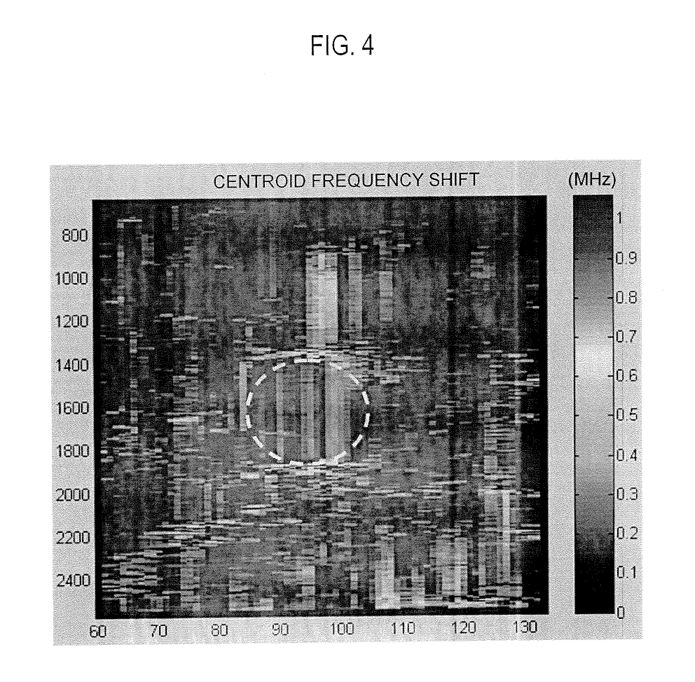

FIG. 4 depicts a center frequency shift image expressed by pixels having pixel values denoted with the central frequency shift between the first and second center frequencies. FIG. 4 shows the result based on the first and second ultrasound echo signals received at a temperature of 40° C. and 70° C., respectively. In FIG. 4, the degree of the central frequency shift is denoted with a pseudo color map. In FIG. 4, a dotted circle denotes the boundary of the lesion. The dotted circle can be expressed with the determined position of the lesion boundary.

Hereinafter, a method of detecting the lesion according to another embodiment of the present invention will be described with reference to FIGS. 1 and 5.

Referring now to FIGS. 1 and 5, the HIFU probe may inject the HIFU on a lesion 210 in a body 200 (step S510). The image probe may transmit ultrasound signals to the lesion when the HIFU is injected onto the lesion 210 (step S520) and receive former and latter ultrasound echo signals from the lesion. The image probe 130 may convert the former and latter ultrasound echo signals into former and latter receive signals (step S530). The signal processor may detect the center frequencies of the former and latter receive signals (step S540) and calculate the center frequency shift between the former and latter receive signals (step S550). The center frequency shift is a pixel value of each pixel for forming a center frequency shift image. After calculating the center frequency shift, the signal processor 150 may search a region including pixels of which pixel values are within a predetermined range and adjacent to one another. Also, the signal processor 150 may determine a position of the lesion based on the center frequency shift (step S560). In case that the signal processor searches the region, the position of the lesion may be determined in consideration of a boundary of the region. The image processor 160 may form the center frequency shift image based on the center frequency shift (step S570), while the display unit may display the center frequency shift image (step S580).

By using the embodiments, thermal diffusion due to injection of the HIFU can be detected.

In the above-described embodiment, the lesion is detected based on the difference of the center frequencies of the first and second ultrasound echo signals, which are obtained before and after the lesion undergoes the HIFU. However, in another embodiment, the lesion may be detected based on the ultrasound echo signals, which are successively obtained when the HIFU is injected onto the lesion, until the lesion is removed. In this embodiment, the center frequencies of former ultrasound receive signals and latter ultrasound receive signals are compared in order to obtain the center frequency shift.

As described above, in accordance with the present invention, the image showing the thermal diffusion due to the temperature difference generated by applying the high intensity focused ultrasound can be provided in real time to the user. Thus, the user can remove the lesion accurately while observing the thermal diffusion in real time during treatment.

An embodiment may be achieved in whole or in parts by an ultrasound diagnostic system, which includes: a high intensity frequency ultrasound (HIFU) probe for injecting the HIFU on a lesion in a body; an imaging probe for transmitting ultrasound signals to the lesion, receiving ultrasound echo signals from the lesion, and outputting receive signals obtained from the ultrasound echo signals; a signal processing unit for detecting center frequencies of two receive signals outputted from the imaging probe and calculating center frequency shift between the two receive signals; an image processor for forming at least one ultrasound image showing the lesion based on the receive signals and a center frequency shift image based on center frequency shift; and a display unit for displaying the ultrasound image and the center frequency shift image.

Another embodiment may be achieved in whole or in parts by an ultrasound diagnostic system, which includes: a high intensity frequency ultrasound (HIFU) probe for injecting the HIFU on a lesion in a body; an imaging probe for transmitting ultrasound signals to the lesion, receiving ultrasound echo signals from the lesion, and outputting receive signals obtained from the ultrasound echo signals; a signal processing unit for detecting center frequencies of two receive signals outputted from the imaging probe, calculating center frequency shift between the two receive signals and determining a position of the lesion in consideration of the center frequency shift; an image processor for forming at least one ultrasound image showing the lesion based on the receive signals and a center frequency shift image based on center frequency shift; and a display unit for displaying the ultrasound image and the center frequency shift image.

An embodiment may be achieved in whole or in parts by a method of detecting a lesion in a body, which includes: transmitting ultrasound signals to a lesion in a body; receiving first ultrasound echo signals from the lesion; injecting high intensity frequency ultrasound (HIFU) on the lesion; transmitting ultrasound signals to a lesion in a body; receiving second ultrasound echo signals from the lesion; obtaining first receive signals from the first ultrasound echo signals and second receive signals from the second ultrasound echo signals; detecting center frequencies of the first and second receive signals; calculating center frequency shift between the first and second receive signals, wherein the center frequency shift is a pixel value of each pixel for forming a center frequency shift image; determining a position of the lesion in consideration of the center frequency shift; forming the center frequency shift image based on the center frequency shift; and displaying the center frequency shift image.

Another embodiment may be achieved in whole or in parts by a method of detecting a lesion in a body, which includes: injectng a high intensity frequency ultrasound (HIFU) on a lesion in a body; transmitting ultrasound signals to the lesion when the HIFU is injected on the lesion; obtaining former and latter receive signals with ultrasound echo signals from the lesion, successively, when the HIFU is injected on the lesion; detecting center frequencies of the former and latter receive signals; calculating center frequency shift between the former and latter receive signals, wherein the center frequency shift is a pixel value of each pixel for forming a center frequency shift image; determining a position of the lesion in consideration of the center frequency shift; forming the center frequency shift image based on the center frequency shift; and displaying the center frequency shift image.

Any reference in this specification to “one embodiment,” “an embodiment,” “example embodiment,” etc., means that a particular feature, structure or characteristic described in connection with the embodiment is included in at least one embodiment of the invention. The appearances of such phrases in various places in the specification are not necessarily all referring to the same embodiment. Further, when a particular feature, structure or characteristic is described in connection with any embodiment, it is submitted that it is within the purview of one skilled in the art to effect such feature, structure or characteristic in connection with other ones of the embodiments.

Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. An ultrasound diagnostic system, comprising:
a high intensity frequency ultrasound (HIFU) probe for injecting the HIFU on a lesion in a body;
an imaging probe for transmitting ultrasound signals to the lesion and receiving ultrasound echo signals from the lesion, the imaging probe further being configured to output receive signals obtained from the ultrasound echo signals;
a signal processing unit for detecting center frequencies of two receive signals outputted from the imaging probe and calculating center frequency shift between the two receive signals;
an image processor for forming at least one ultrasound image showing the lesion based on the receive signals and a center frequency shift image based on center frequency shift; and
a display unit for displaying the ultrasound image and the center frequency shift image.

2. The ultrasound diagnostic system of claim 1, wherein the two receive signals include:
first receive signals obtained by the ultrasound echo signals from the lesion having not undergone the HIFU; and
second receive signals obtained by the ultrasound echo signal from the lesion having undergone the HIFU.

3. The ultrasound diagnostic system of claim 1, wherein the two receive signals include:
former receive signals and latter receive signals successively obtained when the HIFU is injected to the lesion until the lesion is removed.

4. The ultrasound diagnostic system of claim 1, wherein the signal processing unit includes:
a spectrum analyzing unit for analyzing spectra of the two receive signals to produce spectrum analysis results;
a center frequency detecting unit for detecting center frequencies of the two receive signals, respectively, based on the spectrum analysis results; and
a center frequency shift calculating unit for calculating the center frequency shift between the two receive signals.

5. The ultrasound diagnostic system of claim 1, wherein the image processor forms an overlap image of the ultrasound image and the center frequency shift image, and wherein the display unit displays the overlap image.

6. The ultrasound diagnostic system of claim 5, wherein the image processor sets the pixel values of pixels outside a region corresponding to the lesion to zero in the center frequency shift image and forms a transparent center frequency shift image to be overlapped with the ultrasound image.

7. The ultrasound diagnostic system of claim 4, further comprising a container for containing a medium, wherein the contained medium is disposed between the HIFU probe and the body.

8. An ultrasound diagnostic system, comprising:
a high intensity frequency ultrasound (HIFU) probe for injecting the HIFU on a lesion in a body;
an imaging probe for transmitting ultrasound signals to the lesion and receiving ultrasound echo signals from the lesion, the imaging probe further being configured to output receive signals obtained from the ultrasound echo signals;
a signal processing unit for detecting center frequencies of two receive signals outputted from the imaging probe and calculating center frequency shift between the two receive signals, the signal processing unit further being configured to determine a position of the lesion in consideration of the center frequency shift;
an image processor for forming at least one ultrasound image showing the lesion based on the receive signals and a center frequency shift image based on center frequency shift; and
a display unit for displaying the ultrasound image and the center frequency shift image.

9. The ultrasound diagnostic system of claim 8, wherein the signal processing unit includes:
a spectrum analyzing unit for analyzing spectra of the two receive signals to produce spectrum analysis results;
a center frequency detecting unit for detecting center frequencies of the two receive signals, respectively, based on the spectrum analysis results;
a center frequency shift calculating unit for calculating the center frequency shift between the two receive signals; and
a position determining unit for determining the position of the lesion based on the center frequency shift.

10. The ultrasound diagnostic system of claim 9, wherein the center frequency shift is a pixel value of each pixel for forming the center frequency shift image, and wherein the position determining unit searches a region including pixels adjacent to one another, and wherein the pixel values of the pixels in the region are within a predetermined range.

11. The ultrasound diagnostic system of claim 9, wherein the two receive signals include:
first receive signals obtained by the ultrasound echo signals from the lesion having not undergone the HIFU; and
second receive signals obtained by the ultrasound echo signal from the lesion having undergone the HIFU.

12. The ultrasound diagnostic system of claim 9, wherein the two receive signals include:
former receive signals and latter receive signals successively obtained when the HIFU is injected to the lesion until the lesion is removed.

13. The ultrasound diagnostic system of claim 9, wherein the image processor forms an overlap image of the ultrasound image and the center frequency shift image, and wherein the display unit displays the overlap image.

14. The ultrasound diagnostic system of claim 10, wherein the image processor sets the pixel values of pixels outside the region to zero in the center frequency shift image and forms a transparent center frequency shift image to be overlapped with the ultrasound image.

15. The ultrasound diagnostic system of claim 9, further comprising a container for containing a medium, wherein the contained medium is disposed between the HIFU probe and the body.

16. A method of detecting a lesion in a body, comprising:
transmitting ultrasound signals to a lesion in a body;
receiving first ultrasound echo signals from the lesion;
injecting high intensity frequency ultrasound (HIFU) on the lesion;
transmitting ultrasound signals to a lesion in a body;
receiving second ultrasound echo signals from the lesion;
obtaining first receive signals from the first ultrasound echo signals and second receive signals from the second ultrasound echo signals;
detecting center frequencies of the first and second receive signals;
calculating center frequency shift between the first and second receive signals, wherein the center frequency shift is a pixel value of each pixel for forming a center frequency shift image;
determining a position of the lesion in consideration of the center frequency shift;
forming the center frequency shift image based on the center frequency shift; and
displaying the center frequency shift image.

17. The method of claim 16, wherein the first receive signal obtained by the ultrasound echo signals from the lesion having not undergone the HIFU, and wherein the second receive signals obtained by the ultrasound echo signal from the lesion having undergone the HIFU.

18. A method of detecting a lesion in a body, comprising:
injecting a high intensity frequency ultrasound (HIFU) on a lesion in a body;
transmitting ultrasound signals to the lesion when the HIFU is injected on the lesion;
obtaining former and latter receive signals with ultrasound echo signals from the lesion, successively, when the HIFU is injected on the lesion;
detecting center frequencies of the former and latter receive signals;
calculating center frequency shift between the former and latter receive signals, wherein the center frequency shift is a pixel value of each pixel for forming a center frequency shift image;
determining a position of the lesion in consideration of the center frequency shift;
forming the center frequency shift image based on the center frequency shift; and
displaying the center frequency shift image.

19. The method of claim 18, further comprising:
searching a region including pixels adjacent to one another, wherein the pixel values of the pixels in the region are within a predetermined range, and wherein the position of the lesion is determined in consideration of a boundary of the region.
